(19) United States
(12) Patent Application Publication
(10) Pub. No.: US 2002/0083535 A1
(43) Pub. Date: Jul. 4, 2002

(54) Shoe disinfectant and deodorizer

(76) Inventor: Jacob Fraden, La Jolla, CA (US)

Correspondence Address: THE MAXHAM FIRM, 750 "B" STREET, SUITE 3100, SAN DIEGO, CA, 92101, US

(21) Appl. No.: 09/750,412

(22) Filed: Dec. 28, 2000

(51) Int. Cl. A43D 3/00

(52) U.S. Cl. 12/128 B; 12/128 R

(57) ABSTRACT

A device for deodorizing and disinfecting a shoe by means of irradiation of biocidal ultraviolet (UV) light into the shoe interior. The device includes a substantially tubular light pipe that at one end incorporates a UV lamp connected to a power source, while the opposite end of the light pipe is inserted into a shoe. The inserted end may be covered by a protective cover fabricated of a thin polymer film that is substantially transparent in the UV spectral range. The light pipe has a reflective inner surface.

### FIELD OF THE INVENTION

This invention relates to devices for disinfecting and neutralizing odor, and more particularly pertains to disinfecting shoe interior and removing odor by ultraviolet (UV) irradiation.

### DESCRIPTION OF THE PRIOR

The leather of the shoes generally absorbs moisture from, for example, the user's foot, or external conditions such as rain or snow. The moist and warm interior of a shoe also provides ideal conditions for the growth of microorganisms, particularly fungus. Such microorganism and fungus can reside in the shoe, causing unpleasant odor or fungal related problems such as athlete's foot. Shoe borne fungal problems are particularly troublesome because after treatment with drugs, medicated ointment and the like, the foot can be easily re-infected when the user returns to wearing shoes still harboring the fungus. It would be advantageous to have a device which disinfects the interior of the shoe, thereby, preventing re-infections of foot fungus and freshness of the shoe by neutralizing its unpleasant odor. The need for deodorizing shoes is especially important in such cultures, where shoes are customary removed by people before entering the room. This includes the Japanese and Muslim cultures.

Various methods for disinfecting and removing odor from shoes are known in prior art. These include chemical and physical methods. More specifically, chemical methods include various sprays and shoe liners, while physical include irradiation of the shoe interior by UV light. The methods vary by convenience of application, cost, safety, duration of protection and other features. The ultraviolet devices appear to be most efficient, however, they suffer from several disadvantages, including difficulty of a uniform delivery of the UV radiation into the shoe interior, possibility of transmitting infection by a UV lamp housing that is inserted inside the shoe, and a potential for an eye damage by a stray UV irradiation.

By way of example, the prior art includes U.S. Pats. No. 5,829,167 and 5,399,404 for deodorizing shoe linings and U.S. Pat. No. 5,978,996 for the UV disinfecting.

None of known methods of shoe disinfecting and deodorizing provide a complete resolution of the problem. Therefore, it can be appreciated that there exists a continuing need for a new and improved devices for disinfecting and deodorizing shoes. In this regard, the present invention substantially fulfills this need.

It is therefore the goals of this invention are:

provide a device that can safely and efficiently disinfect the interior of a shoe and remove odors;

provide a biocidal light source with a lamp positioned outside of a disinfected area;

provide a protective cover that forms a biological barrier between a shoe and the light source;

Provide a light source with controlled emission field for disinfecting an object.

### SUMMARY OF THE INVENTION

A shoe deodorizer is provided herein for disinfecting the interior of a shoe from microorganism such as fungus. The shoe deodorizer includes an optical light pipe for delivering UV radiation to the shoe interior from a biocidal wavelength light source, such as a UV lamp positioned outside of a shoe, a disposable protective cover, and an electrically operated control apparatus. The electrically operated control apparatus can comprise a timer and safety switches for controlling the operation of a light source.

### BRIEF DESCRIPTION OF THE DRAWINGS

Various embodiments are described below with reference to the drawings wherein:

FIG. 1 is a cross-sectional view of the shoe irradiator.

FIG. 2 is a perspective view of a shoe irradiator inserted into a shoe.

FIGS. 3 and 4 are, respectively, wide and narrow angle irradiators.

FIG. 5 is a view of a portable shoe deodorizer in a folded position.

FIG. 6 is a view of a portable shoe deodorizer in an operational position.

### PREFERRED EMBODIMENTS

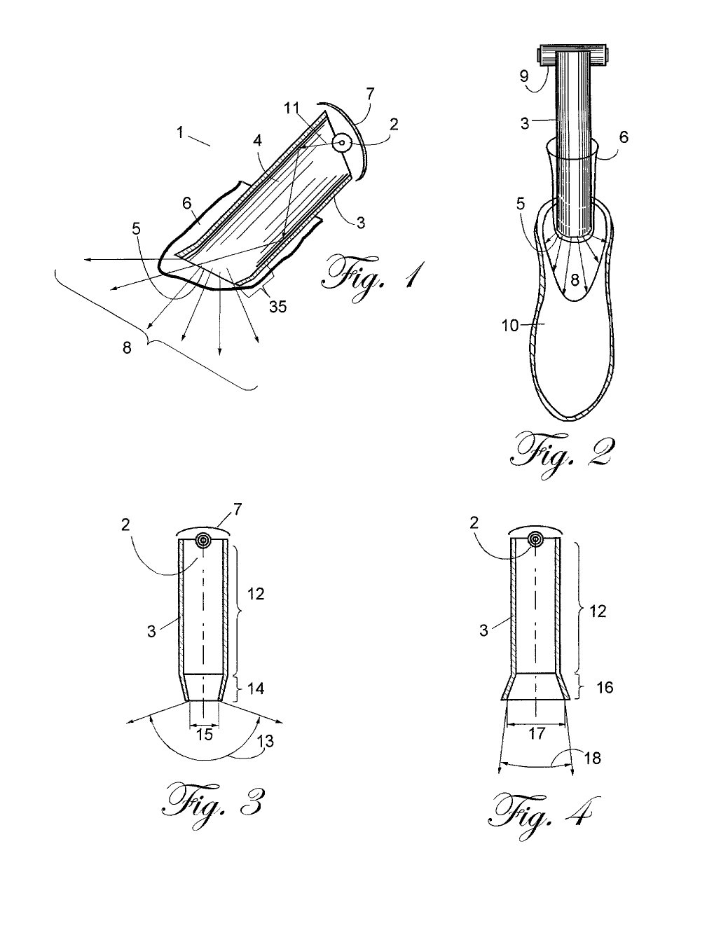

FIG. 1 shows a cross-sectional view of the optical disinfectant/deodorizer 1. The source of biocidal light is lamp 2 that may be a UV lamp. The lamp preferably should emanate at least portion of light in the wavelength range from 2000 to 3000 angstroms. Since most of such lamps emanate light within 360 degree angle, that light needs to be directed toward the desired area. In this embodiment, the light shall be directed into the interior of a shoe and not to any other locations. Moreover, the light shall be diffused within the shoe interior over sufficiently wide area and not to be focused on any particular spot. This is accomplished by light pipe 3 having lamp 2 at one end and the exit aperture 5 at the other end. The lower part of the pipe is to be positioned inside a shoe so that aperture 5 is removed from the shoe inner lining at a distance from about 0.5 to about 2 inches, depending on the shoe size and disinfected area. Lamp 2 may be aided with reflector 7 that diverts radiation toward light pipe 3. Interior 4 of light pipe 3 is made of highly reflective material, such as polished aluminum. Numerous other reflective surfaces are possible that include metallized plastics, films, etc. The device operates as follows. For the illustration purpose, consider ray 11 emitted by lamp 2 toward the interior 4 of light pipe 3. The ray is reflected from the mirror surface of interior 4 in a zigzag manner and exits aperture 5. Different rays 8 exit the light pipe at different angles. FIG. 2 shows light pipe 3 inserted into shoe 10 so that light rays 8 irradiate the shoe interior over a wide area. In FIG. 2, the UV lamp is encased inside light chamber 9. Naturally, devices like the one depicted in FIG. 2 may be stacked one next another for treatment of multiple shoes simultaneously.

There is a possibility that infection from a shoe interior may be transmitted by way of physical contact to the outside surface of a light pipe and, subsequently, transmitted to another shoe or even to the same shoe during the following treatments. This may cause spread of infection or re-infection of the same user. To minimize a risk of such an undesirable event, light pipe 3 may be made disposable and discarded after each shoe treatment. This however, may appear too costly. Alternatively, a disposable protective cover 6 may be used to envelop that portion of light pipe 3 that may come in contact with a shoe. Cover 6 should be fabricated of a material that has a substantial transparency in the wavelength range of the lamp. An example of such a material is polyolefin and more specifically, polyethylene. Thickness of cover 6 should range from 0.0005 to 0.002″ (0.01-0.05 mm). A cover has a shape of a bag in is secured on a light pipe by one of conventional methods known in art. Since such covers can be produced very economically, they can be discarded after each use. It should be appreciated that a protective cover as described above may be used with any type of biocidal light source, including the combination of a lamp and a light pipe, and those devices known in prior art.

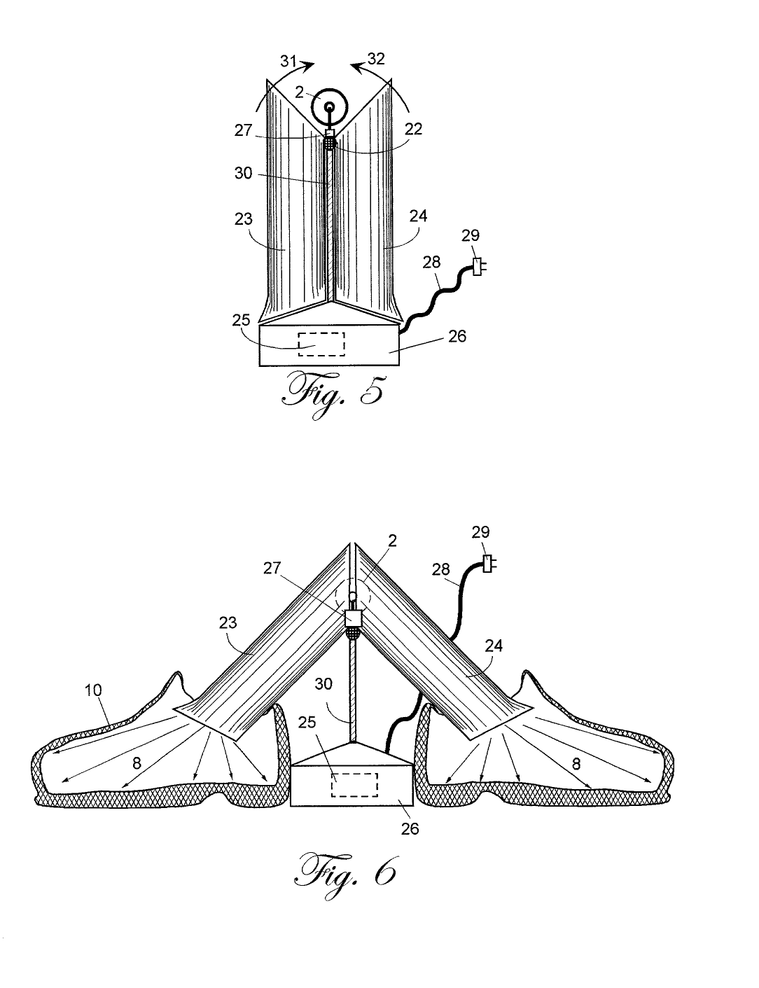

It may be desirable to shape the exit angle of light rays to a desirable value, that is, to make the illuminated field either wider or narrower. This can be achieved by varying the substantially circular profile of a light pipe along the light pipe length, especially at its portion that is inserted into a shoe. FIGS. 3 and 4 illustrate that the exit angle can be made either very wide (13) or very narrow (18) by shaping the lower portion of the light pipe. Lower portions 14 and 16 of light pipes 3 have conical shapes, where the smaller diameter 15 leads to wider angle 13, while the larger diameter 17 results in narrower angle 18. For a more efficient light distribution, lower portions 14 and 16 may have a parabolic, rather than linear profile. As a practical matter, it is usually desirable to irradiate the vamp portion of a show. To deliver light better to that area, the light pipe may have a slightly curved shape of the lower portion 35, as illustrated in FIG. 1. FIGS. 5 and 6 illustrate a portable device produced according to this invention. It is depicted in FIG. 6 in a folded non-operational state. The device is comprised of two light pipes 23 and 24, biocidal lamp 2, switch 27, pivot 22, supporting pole 30 and base 26. In turn, base 26 may contain electronic module 25 and electric cord 28 with plug 29. In this folded state, switch 27 disables lamp 2 in order to prevent eye damage if the device is activated prematurely. To operate, the light pipes are rotated around pivot 22 in respective directions 31 and 32, until lamp 2 is fully enclosed inside both light pipes, as shown in FIG. 6. The entire structure is supported by pole 30 attached to base 26. At the operational position position, switch 27 enables lamp that may be now activated by the electronic module. Each light pipe is inserted into shoe 10. When powered, lamp 2 emanates light rays into both light pipes 23 and 24, from which light rays 8 exit into the interior of a respective shoe 10. Electronic module 25 may contain a power supply, power switch and a timer that turns light off after the treatment procedure is complete. Practically, the time of treatment ranges from 2 to 10 minutes, depending on the power of lamp 2, geometry and reflectivity of light pipes 23 and 24, protective covers, if any, and shoe sizes. In addition, electronic module 25 may contain components required for operation of lamp 2, such as an electrical ballast. The ballast provides the voltage required by lamp 2, usually a stepped-up voltage.

While the above description contains many specifics, these specifics should not be construed as limitations on the scope of the invention, but merely as exemplifications of preferred embodiments thereof Those skilled in the art will envision many other possible variations that are within the scope and spirit of the invention as defined by the claims appended hereto.

1. A device for disinfecting and deodorizing by way of irradiation by biocidal light, comprising in combination at least one of each:
a lamp,
a light pipe having reflective inner surface, and power source.

2. A device of claim 1 wherein said lamp emanates at least portion of light in an ultraviolet spectral range.

3. A device of claim 1 wherein said light pipe is made of metallized plastic.

4. A device of claim 1 wherein said light pipe has a curvature in at least a portion of its length.

5. A device of claim 1 wherein said light pipe has a substantially circular cross-sectional profile.

6. A device of claim 4 wherein said circular profile varies in size along at least portion of the length of said light pipe.

7. A device for disinfecting and deodorizing of a shoe interior, containing a biocidal light source and a protective cover enveloping at least that portion of light source that may come in contact with a shoe.

8. A device for disinfecting and deodorizing of a shoe interior of claim 7 wherein said cover is made of a polyolefin film.

9. A device for disinfecting and deodorizing of a shoe interior of claim 7 wherein said film has thickness ranging from 0.0005 to 0.002 inch.

10. A device of claim 7 wherein said light source is comprised of an ultraviolet lamp and a light pipe.
